(19) United States
(12) Patent Application Publication
(10) Pub. No.: US 2011/0295163 A1
(43) Pub. Date: Dec. 1, 2011

(54) Therapeutic hypothermia and cardio-respiratory augmentation apparatus

(76) Inventor: R. Vijayanagar, Tampa, FL (US)

(21) Appl. No.: 12/927,745

(22) Filed: Nov. 23, 2010

Related U.S. Application Data
(60) Provisional application No. 61/281,909, filed on Nov. 24, 2009.

(51) Int. Cl. A61H 31/00; A61F 7/00

(52) U.S. Cl. 601/18; 601/44

(57) ABSTRACT

A therapeutic hypothermia and cardio-respiratory augmentation apparatus to cool a patient to a selected temperature range and augment the patient's cardio-pulmonary functions comprising a cooling system including an upper cooling section, an intermediate cooling section and a lower cooling section coupled to a fluid cooling source by a cooling fluid circulating system to supply a cooling fluid to the upper cooling section, the intermediate cooling section and the lower cooling section to selectively and independently cool the brain, the torso and the legs respectively to within a selected or predetermined temperature range, a cardio-respiratory augmentation system including an upper biphasic ventilator section and a lower pulmonary augmentation section coupled to a gas source by a gas circulating system to alternatively apply positive pressure and negative pressure to the upper biphasic ventilator section and pulsating positive pressure to the lower pulmonary section to augment respiration for the lungs and blood flow to the heart respectively and a control system including a plurality of sensors to sense the patient's temperature, pulse rate and respiration rate and logic circuitry to control the operation of the cooling system and the cardio-respiratory augmentation system.

### CROSS-REFERENCE

This application claims priority from pending provisional patent application Ser. No. 61/281,909 filed Nov. 24, 2009.

### BACKGROUND OF THE INVENTION

1. Field of the Invention

A therapeutic hypothermia and cardio-respiratory augmentation apparatus to cool a patient to within a selected or predetermined temperature range and augment the patient's cardio-pulmonary functions.

2. Description of the Prior Art

Various devices have been developed to assist a patient's breathing comprising either internal or external ventilators.

Internal ventilator systems commonly direct a flow of gas into the lungs through a face mask or intubation. An alternating gas pressure is used to create a tidal flow of gas into and out of the patient's lungs.

Early external ventilators known as iron lungs comprise various forms of enclosure to encase the torso of a patient. Alternating gas pressure is applied to the interior of the enclosure to produce compression and expansion of the chest and to produce a tidal flow of air into and out of the lungs.

A more modern example of an external ventilation is known as a biphasic cuirass ventilation which requires the patient to wear an upper body shell. The ventilation is biphasic because the upper body shell is coupled to a pump which controls both the inspiratory and expiratory phases of the respiratory cycle. U.S. Pat. No. 6,345,618 discloses such a cuirass type ventilator comprising a shell including a peripheral edge having a sealing device secured thereto. The sealing device comprises a sealing member depending from the peripheral edge of the shell having a sealing region for sealing against a patient's body, a securing region where the sealing member is secured to the shell and a resilient pleated region intermediate the sealing region and securing region such that in use, the resilience of the pleated region urges the sealing region into sealing engagement with a patient's body.

U.S. Pat. No. 5,988,166 describes a ventilator apparatus for ventilating the lungs of a patient comprising a source of gas pressure applied internally and externally to the lungs. The alternating pressure has a complex waveform deconstructable into a large amplitude waveform of a first frequency which may be produced by a blower and a valve and a lower amplitude waveform of a higher frequency which may be produced by a second blower and valve unit.

In addition, numerous systems and devices have been designed to selectively cool various organs of the human body such as the brain, kidney, and heart to within a temperature range. The induction of mild hypothermia lowering a patient's temperature from 37° C. to between 32° and 35° C. after cardiac arrest was proposed as early as the 1950s in an effort to protect the brain against global ischemia.

For example, therapeutic hypothermia is utilized to protect the brain from injury. Cooling of the brain is often accomplished through whole body cooling or total body hypothermia. This cooling can be accomplished by immersing the patient in ice, by using cooling blankets or by cooling the blood flowing externally through a cardiopulmonary bypass machine such as found in U.S. Pat. No. 3,425,419 or U.S. Pat. No. 5,486,208.

Medivance, Inc, of Louisville, Colo. has developed a non-invasive patient cooling system that controls, monitors and maintains core body temperature to induce mild hypothermia. Such a system comprises a control module and energy transfer pads. Using standard temperature probes, a clinician controls a patient's temperature within a range of 33° to 37° C. The system senses the patient's temperature and compares the temperature to a preset target temperature programmed before the procedure begins. The system then adjusts the temperature of circulating water through the energy transfer pads using a proprietary thermoregulatory algorithm.

The Medivance energy transfer pads comprise three layers covering a major portion of the patient's body surface area including an inner hydrogel layer, a middle thin film layer of polyolefin film and an outer foam layer of compression-molded foam. Water is circulated through the pads, simulating immersion of the body in cool water.

Microprocessors control and monitor operation of the Medivance system. Each microprocessor monitors independent water and patient temperatures as well as other parameters determined by the clinician. Additionally, there are dual patient temperature sensor inputs. If a fault occurs, each processor has full control to stop therapy delivery. The system monitors the patient temperature. If the temperature exceeds 38° C. or falls below 32° C. the microprocessors will stop the therapy.

U.S. Pat. No. 6,682,552 teaches a device and system to cool the brain comprising a cold insert into an arch that fits around the neck of a patient, without obstructing the airway of the subject. A cold insert, such as a frozen fluid or endothermic packet, is disposed within one or both of the terminal ends of the arch, adjacent to one or both of the subject's carotid arteries. The cooling effect is specifically directed to cool the blood flowing through the carotid artery to the brain. A system incorporating the device with a temperature monitor is also disclosed. The system allows a health care professional to monitor and regulate cooling as required in the pre-hospital setting.

Another system from InnerCool comprises a non-invasive surface pad cooling system consisting of a console connected to surface pads that are applied to the patient's torso and upper thighs. The console regulates water circulation through the pads to achieve and maintain desired patient temperature.

CSZ Medical has designed a whole body hypothermia system as an effective alternative to traditional patient cooling techniques. This system combines a head wrap, patient vest and lower body blanket for a non-invasive whole body hypothermia system.

Although there are numerous therapeutic hypothermia systems and various cardio respiratory augmentation devices, there remains a need for an integrated system to combine the benefits of these two (2) medical advances.

### SUMMARY OF THE INVENTION

The present invention relates to a non-invasive method and apparatus for neuro-cardiac protection using therapeutic hypothermia in combination with cardiopulmonary or cardio-respiratory augmentation.

The therapeutic hypothermia and cardiopulmonary or respiratory augmentation apparatus comprises a cooling system to cool the patient and a cardio-respiratory augmentation system to augment ventilation of the patient's lungs and circulation of the blood.

The cooling system for the application of therapeutic hypothermia comprises a fluid cooling source including a fluid pump and refrigeration device coupled to cooling pads, caps, hood vests or similar devices placed against or in heat transfer relationship to the patient's head, torso and legs to circulate cooling fluid or coolant from the fluid cooling source to the patient. The cooling system also includes a plurality of temperature sensors coupled to the fluid cooling source to sense the temperature of the patient and generate a corresponding temperature signal fed to the fluid cooling source. A control module including a timer or clock and logic circuitry to control the operation of the fluid cooling source may be physically incorporated into the fluid cooling source or comprise a separate unit coupled to the fluid cooling source.

The cardio-respiratory augmentation system comprising a pneumatic pressure device including at least one pump device with control valves coupled to an upper ventilator section by a ventilator conduit or hose and coupled to a lower pulmonary augmentation section by a pulmonary augmentation conduit or hose to independently supply air or gas to the upper ventilator section and the lower pulmonary augmentation section. The cardio-respiratory augmentation system also includes a plurality of sensors coupled or mounted on or to the upper ventilator section and the lower pulmonary augmentation section to sense specific patient functions and create a corresponding signal fed to the pneumatic pressure device. In particular, a heart beat sensor and a respiratory rate sensor are mounted to or over the upper ventilator section to monitor the heart rate and breathing rate respectively and a pulse rate sensor is attached to the lower pulmonary augmentation section to monitor the pulse rate in the lower leg portion of the patient operatively coupled to the pneumatic pressure apparatus by corresponding conductors to feed corresponding signals thereto. A control module including a timer or clock and logic circuitry to control the operation of the pneumatic pressure apparatus may be physically incorporated into the pneumatic pressure device or comprise a separate unit coupled to the pneumatic pressure device.

The upper ventilator section comprises a body shell coupled to the pneumatic pressure device by the ventilator conduit or hose through a port formed in the body shell. The lower pulmonary augmentation section comprises a plurality of air or gas passages formed between the two fabric layers coupled to the pneumatic pressure device through the pulmonary augmentation conduit or hose at the lower end portions thereof and a plurality of valves.

The body shell of the upper ventilator section and the pump of the pneumatic pressure device operate as a biphasic cuirass ventilator under the control of the control module pumping air out of the body shell creating a negative pressure around the chest or torso and during the inspiratory phase of the respiratory cycle and creating a positive pressure around the chest or torso during the expiratory phase of the respiratory cycle.

The plurality of air or gas passages with corresponding valves and the pump of the pneumatic pressure device operate to expand the air or gas passages during the diastrophic action of the heart to force blood up and through the veins in the legs and abdomen toward the heart.

The control modules synchronize the operation of the upper ventilator section with the patient's breathing and the lower pulmonary augmentation section with the patient's heart rate.

The invention accordingly comprises the features of construction, combination of elements, and arrangement of parts which will be exemplified in the construction hereinafter set forth, and the scope of the invention will be indicated in the claims.

### BRIEF DESCRIPTION OF THE DRAWINGS

For a fuller understanding of the nature and object of the invention, reference should be had to the following detailed description taken in connection with the accompanying drawings in which:

FIG. 1 illustrates the location of various pressure and temperature sensors in relationship to a patient's body.

FIG. 2 is a top view of a cooling system of the present invention.

FIG. 3 is a side view of the cooling system of the present invention shown in FIG. 2.

FIG. 4 is a top view of an alternate embodiment of the cooling system of the present invention.

FIG. 5 is a side view of the alternate embodiment of the cooling system of the present invention shown in FIG. 4.

FIG. 6 is a front view of a cardio-respiratory augmentation system of the present invention.

FIG. 7 is a side view of a therapeutic hypothermia and cardio-respiratory augmentation apparatus of the present invention.

FIG. 8 is a front view of the therapeutic hypothermia and cardio-respiratory augmentation apparatus of the present invention shown in FIG. 7.

FIG. 9 is a partial detail view of the cooling pants and lower coolant ducting of the therapeutic hypothermia and cardio-respiratory augmentation apparatus of FIGS. 8 and 9.

FIG. 10 is a partial cross-section top view of the cooling pants and lower coolant ducting of the therapeutic hypothermia and cardio-respiratory augmentation apparatus of FIGS. 8 and 9.

FIG. 11 is a partial cross-section top view of the cooling pants and lower coolant ducting of the therapeutic hypothermia and cardio-respiratory augmentation apparatus of FIGS. 8 and 9 during the cardio-respiratory augmentation phase.

Similar reference characters refer to similar parts throughout the several views of the drawings.

### DETAILED DESCRIPTION OF THE PREFERRED EMBODIMENT

The present invention relates to a non-invasive method and apparatus for neuro-cardiac protection using therapeutic hypothermia in combination with cardiopulmonary or cardio-respiratory augmentation.

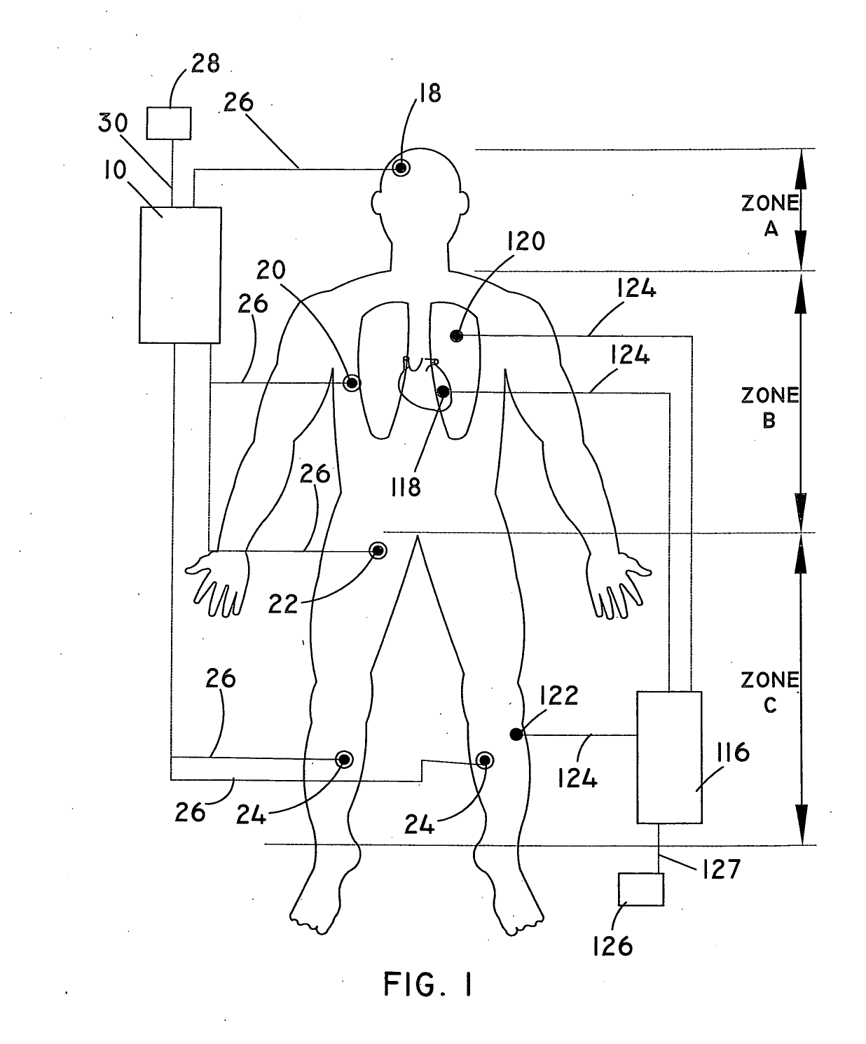

FIG. 1 illustrates the outline of a patient separated in three (3) treatment zones, ZONE A, ZONE B and ZONE C. ZONES A, B and C are selectively treatable with therapeutic hypothermia; while ZONES B and C are selectively treatable with cardiopulmonary or cardio-respiratory augmentation as described hereinafter.

The therapeutic hypothermia and cardiopulmonary or respiratory augmentation apparatus comprises a cooling system to selectively and independently cool each of the three (3) zones and a cardio-respiratory augmentation system to selectively and independently augment ventilation of the patient's lungs (ZONE B) and circulation of the blood to the patient's heart (ZONE C).

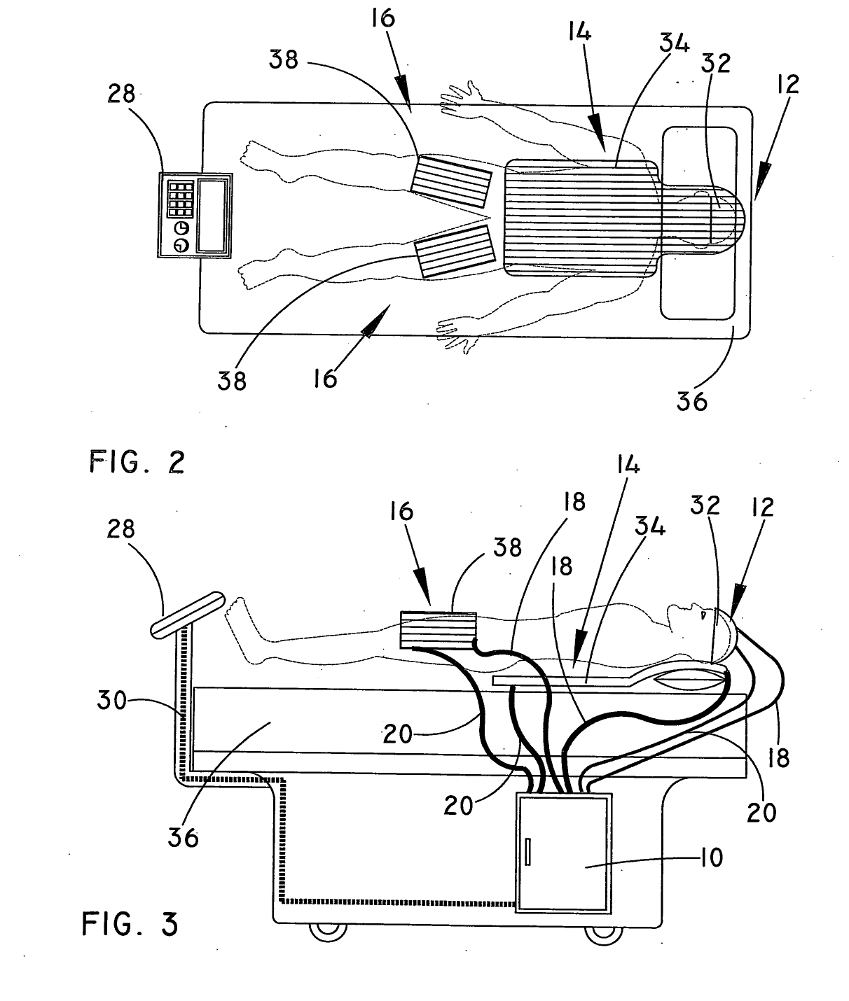

As shown in FIGS. 2 and 3, FIGS. 4 and 5, and FIGS. 7 and 8, the cooling system for the application of therapeutic hypothermia comprises a fluid cooling source 10 including a fluid pump and refrigeration device coupled to an upper cooling section generally indicated as 12, an intermediate cooling section generally indicated as 14, and a lower cooling section generally indicated as 16 by a fluid supply conduit or hose 18 and a fluid return conduit or hose 20 to circulate cooling fluid or coolant from the fluid cooling source 10 to the upper cooling section 12, intermediate cooling section 14 and lower cooling section 16 and return to the fluid cooling source 10 to independently cool each of the three (3) zones as described more fully hereinafter. The cooling system also includes a plurality of temperature sensors coupled to the fluid cooling source 10 to sense the temperature of the patient within each zone and generate a corresponding temperature signal fed to the fluid cooling source 10. Specifically the temperatures of the brain area (ZONE A), the body or torso area (ZONE B), hip/abdomen area (ZONE C) and the leg area (ZONE C) are sensed by temperature sensors 18, 20, 22 and 24 respectively operatively coupled to the fluid cooling source 10 by corresponding conductors 26 to feed corresponding temperature signals thereto. A control module 28 including a timer or clock and logic circuitry to control the operation of the fluid cooling source 10 may be physically incorporated into the fluid cooling source 10 or comprises a separate unit coupled to the fluid cooling source 10 by a cable 30 as shown in the figures.

As shown in FIGS. 2 and 3, the upper cooling section 12 comprises a cap or hood 32, while the intermediate cooling section 14 comprises a body or torso pad 34 supported on or built into a bed 36 and the lower cooling section 16 comprises a pair of thigh pads each indicated as 38.

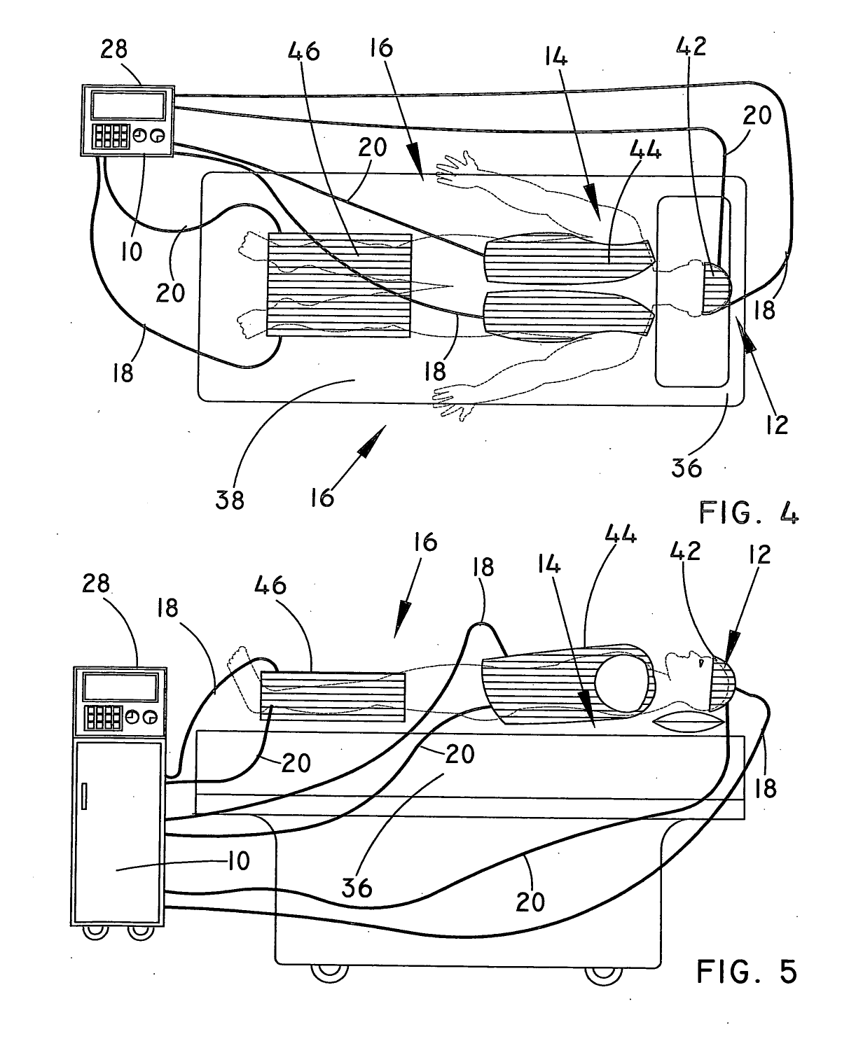

As shown in FIGS. 4 and 5, the upper cooling section 12 comprises a cap or head wrap 42, while the intermediate cooling section 14 comprises a body or torso vest 44 and the lower cooling section 16 comprises a leg blanket 46.

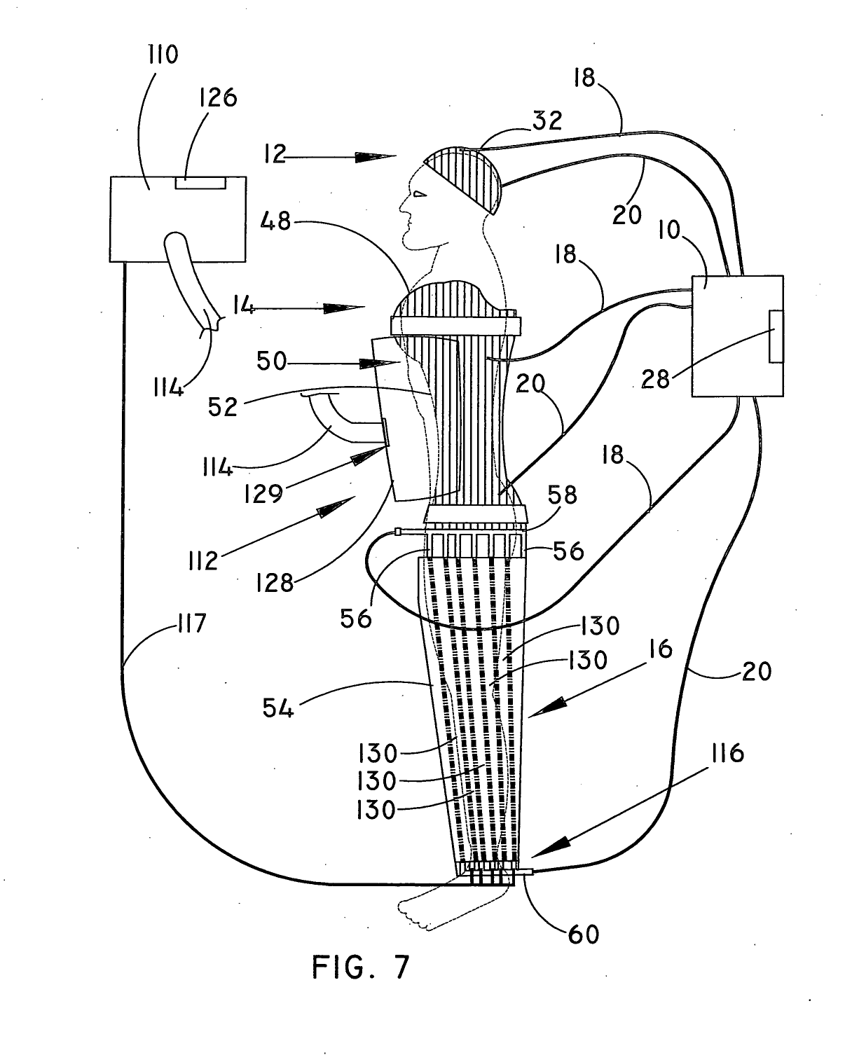

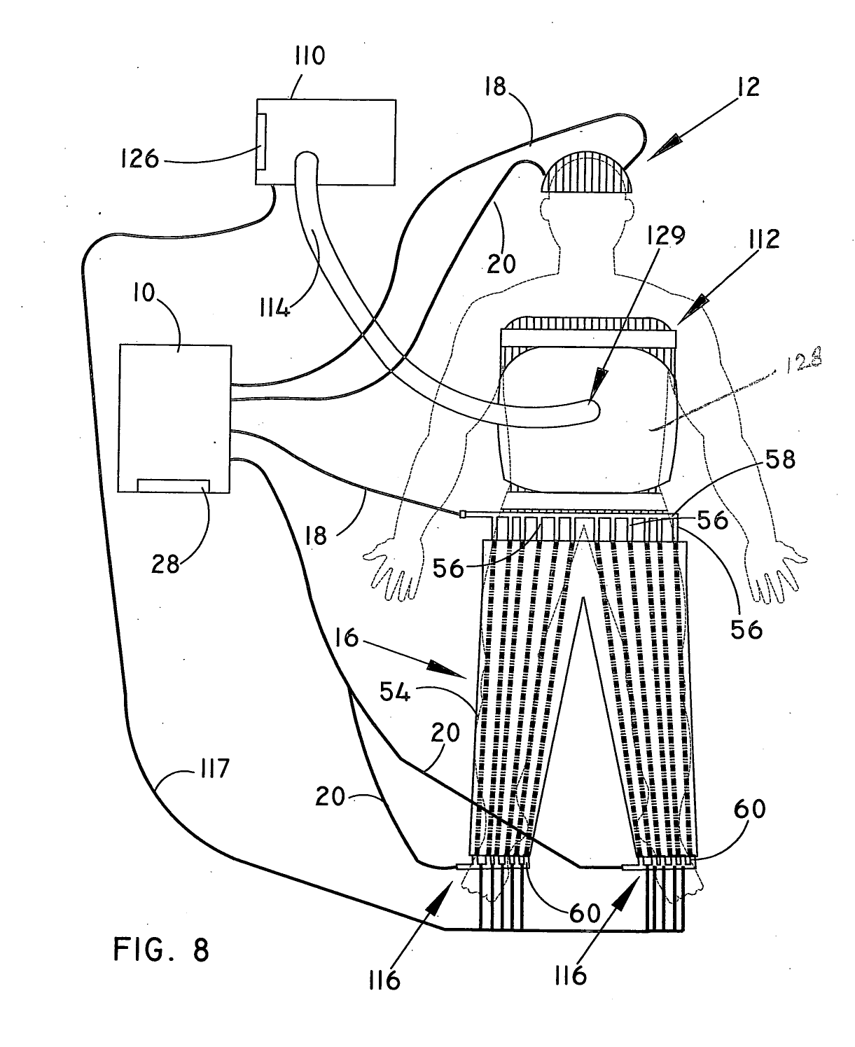

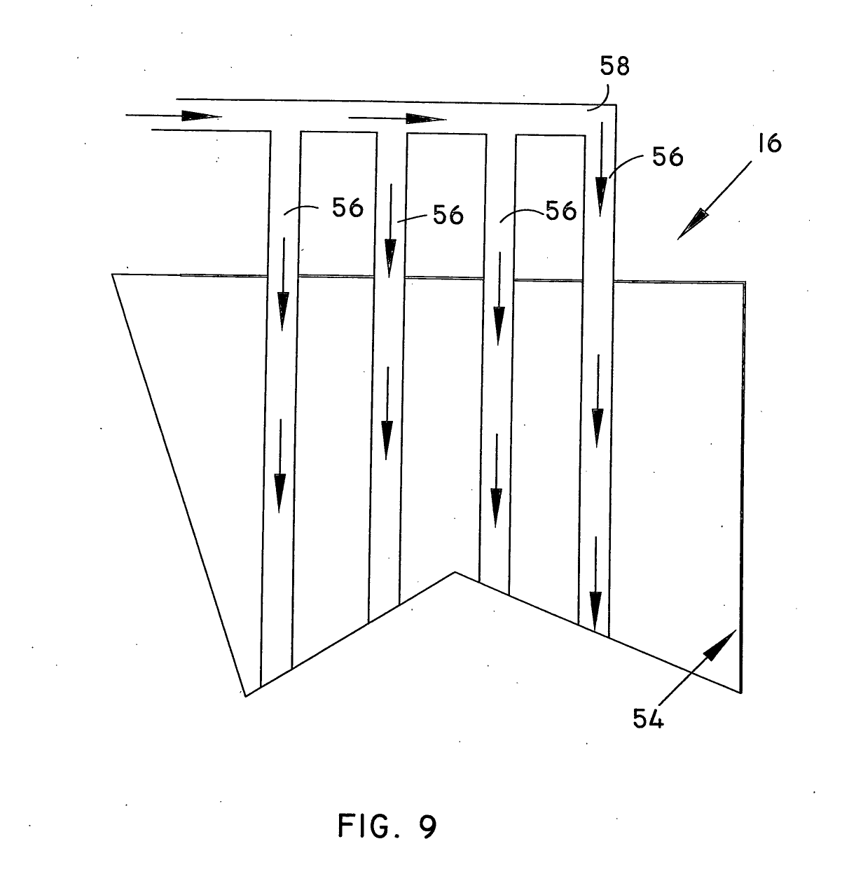

As shown in FIGS. 7 and 8, the upper cooling section 12 comprises a cap or hood 32, while the intermediate cooling section 14 comprises a body or torso vest 48 that may include an open chest area 50 to accommodate a portion of the cardio-respiratory augmentation system as described hereinafter and the lower cooling section 16 comprises cooling pants 54 including lower coolant ducting as best shown in FIGS. 9 through 11. The body or torso vest 48 may include a thin membrane 52 to cover the open chest area 50 without interfering in the operation of the cardio-respiratory augmentation system as described hereinafter. The lower coolant ducting comprises a plurality of lower coolant conduits or hoses each indicated as 56 extending between an upper inlet or supply coolant manifold 58 to be disposed about the waist area of the patient and a pair of lower outlet or return coolant manifolds each indicated as 60 to be disposed about the ankle areas of the patient to circulate cooling fluid or coolant between the lower cooling section 16 and the fluid cooling source 10 through the fluid conduits 18 and 20.

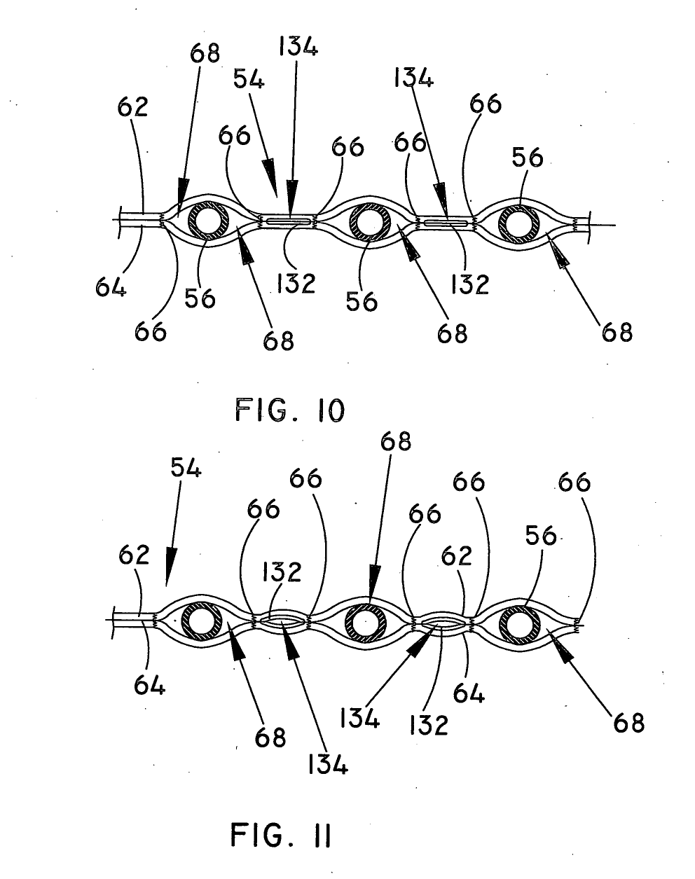

As best shown in FIGS. 10 and 11, the cooling pants 54 comprise an outer layer of spandex-like material 62 and an inner layer of relatively soft material 64, sewn together substantially the length of the cooling pant 54 at stitching 66 to create a plurality of pockets each indicated as 68 to receive a corresponding lower coolant conduit or hose 56. The upper inlet or supply coolant manifold 58 and lower outlet or return coolant manifold 60 may be sewn into the cooling pants 54.

The rate of cooling resulting from the cooling system is a function of the temperature and rate of the fluid or cooling, heat transfer coefficients of the cooling components and surface area contacting or in close proximity to the patient.

As the temperature sensors 18, 20, 22 and 24 feed real time temperature data to the control module 28, the logic circuitry compares the temperature data to a selected temperature range such as between 32° C. and 35° C. and generates control signals to control the temperature and/or flow rate of the coolant circulated to the cooling sections 12, 14 and/or 16.

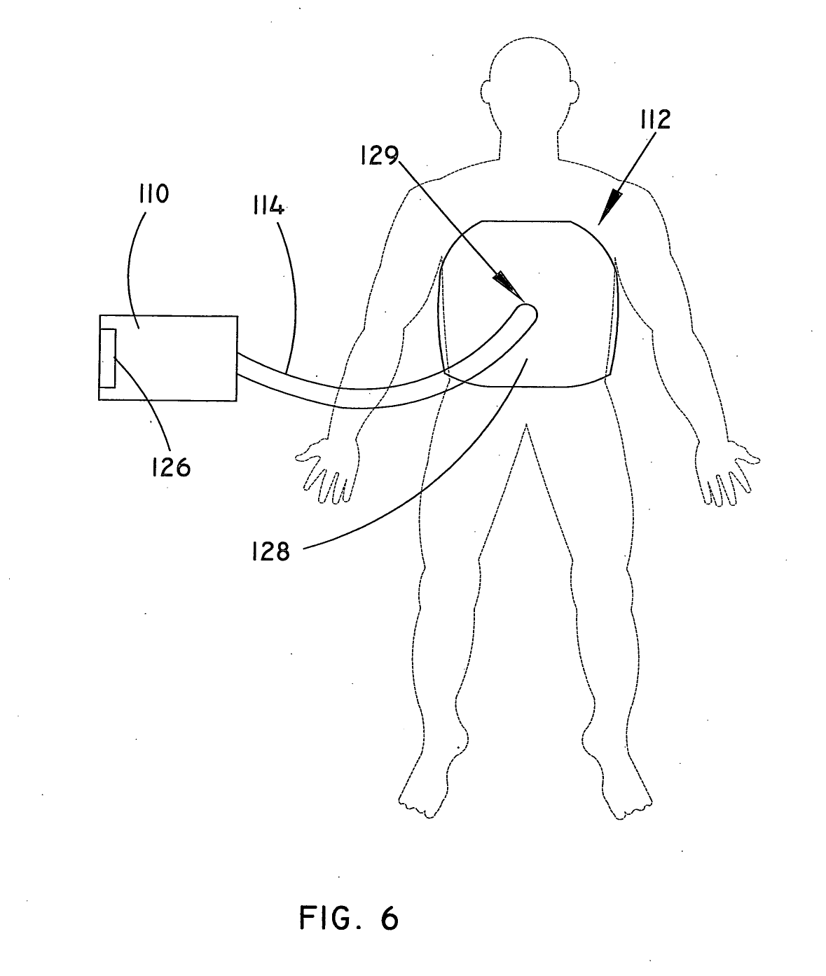

FIGS. 6 through 8, 10 and 11 illustrate the cardio-respiratory augmentation system comprising a pneumatic pressure device 110 including at least one (1) pump device with control valves coupled to an upper ventilator section generally indicated as 112 by a ventilator conduit or hose 114 and coupled to a lower pulmonary augmentation section by a pulmonary augmentation conduit or hose 116 to independently supply air or gas to the upper ventilator section 112 and the lower pulmonary augmentation section 116 as described more fully hereinafter. The cardio-respiratory augmentation system also includes a plurality of sensors coupled or mounted on or to the upper ventilator section 112 and the lower pulmonary augmentation section 116 to sense specific patient functions and create a corresponding signal fed to the pneumatic pressure device 110. In particular, as shown in FIG. 1, a heart beat sensor 118 and a respiratory rate sensor 120 are mounted to or over the upper ventilator section 110 to monitor the heart rate and breathing rate respectively and a pulse rate sensor 122 attached to the lower pulmonary augmentation section 116 to monitor the pulse rate in the lower leg portion of the patient operatively coupled to the pneumatic pressure apparatus 110 by corresponding conductors 124 to feed corresponding signals thereto. A control module 126 including a timer or clock and logic circuitry to control the operation of the pneumatic pressure device 110 may be physically incorporated into the pneumatic pressure device 110 or comprise a separate unit coupled to pneumatic pressure device 110 by a cable 127 as shown in the figures.

As best shown in FIGS. 6 through 8, the upper ventilator section 112 comprises a body shell 128 coupled to the pneumatic pressure device 110 by the ventilator conduit or hose 114 through a port 129 formed in the body shell 128. The lower pulmonary augmentation section 116 comprises a plurality of air or gas passages or sleeves each indicated as 130 formed between the two (2) fabric layers 60 and 62 of the pants 54 by the stitching 66 that also forms adjacent pockets 62 coupled to the pneumatic pressure device 110 through the pulmonary augmentation conduit or hose 117 at the lower end portions thereof and a plurality of resilient, flexible or expandable valves each indicated as 132 each including an outlet slit or aperture 134 formed therein disposed at the upper end portion of each the air or gas passages or sleeves 130.

The body shell 128 of the upper ventilator section 112 and the pump of the pneumatic pressure device 110 operate as a biphasic cuirass ventilator under the control of the control module 126 pumping air out of the body shell 128 creating a negative pressure around the chest or torso during the inspiratory phase of the respiratory cycle and creating a positive pressure around the chest or torso during the expiratory phase of the respiratory cycle.

The plurality of air or gas passages or sleeves 130 with corresponding valves 132 and the pump of the pneumatic pressure device 110 operate to expand the air or gas passages 130 during the diastrophic action of the heart to force blood up and through the veins in the legs and abdomen toward the heart. The cardio-respiratory augmentation system may include separate pumps for the upper ventilator section 112 and the lower pulmonary augmentation section 116 or a valving arrangement to selectively and independently control the function thereof from single pump.

The control module 126 synchronizes the operation of the upper ventilation section 112 with the patient's breathing and the lower pulmonary augmentation section 116 with the patient's heart rate.

It will thus be seen that the objects set forth above, among those made apparent from the preceding description are efficiently attained and since certain changes may be made in the above construction without departing from the scope of the invention, it is intended that all matter contained in the above description or shown in the accompanying drawing shall be interpreted as illustrative and not in a limiting sense.

What is claimed is:

1. A cardio-respiratory augmentation apparatus to augment the patient's cardio-pulmonary functions comprising a cardio-respiratory augmentation system including an upper biphasic ventilator section and a lower pulmonary augmentation section coupled to a gas source by a gas circulating system to alternately apply positive pressure and negative pressure to said upper biphasic ventilator section and pulsating positive pressure to said lower pulmonary section to augment respiration for the lungs and blood flow to the heart respectively and a control system including a plurality of sensors to sense the patient's pulse rate and respiration rate and logic circuitry to control the operation of said cardio-respiratory augmentation system.

2. The cardio-respiratory augmentation apparatus of claim 1 comprising a pneumatic pressure device including at least one pump device with control valves coupled to said upper ventilator section by a ventilator conduit and coupled to said lower pulmonary augmentation section by a pulmonary augmentation conduit to independently supply gas to said upper ventilator section and said lower pulmonary augmentation section.

3. The cardio-respiratory augmentation apparatus of claim 2 wherein said cardio-respiratory augmentation plurality of sensors are coupled to said upper ventilator section and said lower pulmonary augmentation section to sense specific patient functions and create a corresponding signal fed to said pneumatic pressure device.

4. The cardio-respiratory augmentation apparatus of claim 3 wherein said plurality of sensors comprises a heart beat sensor and a respiratory rate sensor mounted to said upper ventilator section to monitor the heart rate and breathing rate respectively and a pulse rate sensor attached to said lower pulmonary augmentation section to monitor the pulse rate in the lower leg portion of the patient operatively coupled to said pneumatic pressure device by corresponding conductors to feed corresponding signals thereto.

5. The cardio-respiratory augmentation apparatus of claim 2 wherein said control system comprises a control module including a timer and logic circuitry to control said operation of said pneumatic pressure device.

6. The cardio-respiratory augmentation apparatus of claim 2 wherein said upper ventilator section comprises a body shell coupled to said pneumatic pressure device by said ventilator conduit through a port formed in said body shell and said lower pulmonary augmentation section comprises a plurality of gas passages formed between fabric layers of said pants.

7. The cardio-respiratory augmentation apparatus of claim 6 wherein said pants further include pockets coupled to the pneumatic pressure device through said pulmonary augmentation conduit at the lower end portions thereof having a plurality of valves each including an outlet aperture formed therein disposed at the upper end portion of each said gas passages.

8. The cardio-respiratory augmentation apparatus of claim 6 wherein said body shell of said upper ventilator section and said pump of said pneumatic pressure device operate as a biphasic cuirass ventilator controlled by said control module pumping air out of said body shell creating a negative pressure around the chest or torso during the inspiratory phase of the respiratory cycle and creating a positive pressure around the chest or torso during the expiratory phase of the respiratory cycle.

9. The cardio-respiratory augmentation apparatus of claim 8 wherein said plurality of gas passages with corresponding valves and said pump of said pneumatic pressure device operate to expand said gas passages during the diastrophic action of the heart to force blood up and through the veins in the legs and abdomen toward the heart.

10. The cardio-respiratory augmentation apparatus of claim 1 wherein said control module synchronizes the operation of said upper ventilation section with the patient's breathing and said lower pulmonary augmentation section with the patient's heart rate.

11. A therapeutic hypothermia and cardio-respiratory augmentation apparatus to cool a patient to a selected temperature range and augment said patient's cardio-pulmonary functions comprising a cooling system including an upper cooling section, an intermediate cooling section and a lower cooling section coupled to a fluid cooling source by a cooling fluid circulating system to supply a cooling fluid to said upper cooling section, said intermediate cooling section and said lower cooling section to selectively and independently cool the brain, the torso and the legs respectively to within a selected or predetermined temperature range, a cardio-respiratory augmentation system including an upper biphasic ventilator section and a lower pulmonary augmentation section coupled to a gas source by a gas circulating system to alternately apply positive pressure and negative pressure to said upper biphasic ventilator section and pulsating positive pressure to said lower pulmonary section to augment respiration for the lungs and blood flow to the heart respectively and a control system including a plurality of sensors to sense the patient's temperature, pulse rate and respiration rate and logic circuitry to control the operation of said cooling system and the cardio-respiratory augmentation system.
